(19) United States
(12) Patent Application Publication
(10) Pub. No.: US 2006/0122588 A1
(43) Pub. Date: Jun. 8, 2006

(54) Cryosurgery compositions and methods

(75) Inventors: John C. Bischof, St. Paul, MN (US); Bumsoo Han, Minneapolis, MN (US)

Correspondence Address: MUETING, RAASCH & GEBHARDT, P.A., P.O. BOX 581415, MINNEAPOLIS, MN, 55458, US

(73) Assignee: Regents of the University of Minnesota, Minneapolis, MN (US)

(21) Appl. No.: 11/340,347

(22) Filed: Jan. 26, 2006

Related U.S. Application Data
(62) Division of application No. 10/461,763, filed on Jun. 13, 2003.
(60) Provisional application No. 60/388,223, filed on Jun. 13, 2002.

(30) Foreign Application Priority Data
Jun. 13, 2003 (WO) PCT/US03/18984

(51) Int. Cl. A61B 18/04

(52) U.S. Cl. 606/20; 606/21

(57) ABSTRACT

A eutectic changing composition, including a system and method of its use. The eutectic changing composition can be used in a localized area of a biological material, such as in a mammal, where the eutectic changing composition includes as an active ingredient at least one solute effective to change a tissue eutectic freezing point at the localized area of biological material. The solute can be effective to increase the tissue eutectic freezing point of the biological material.

### CROSS-REFERENCE TO RELATED APPLICATIONS

This application claims priority from U.S. Provisional Application Ser. No. 60/388,223, filed Jun. 13, 2002, the entire content of which is incorporated herein by reference.

### GOVERNMENT FUNDING

The present invention was made with government support under Grant No. BES 9703326, awarded by the National Science Foundation, and grant No. 5R29CA75284-05, awarded by the National Institutes of Health. The Government may have certain rights in this invention.

### TECHNICAL FIELD

The present invention relates generally to cryosurgery.

### BACKGROUND OF THE INVENTION

Cryosurgery is a minimally invasive surgery technique in which malignant tissue is destroyed by freezing. During a cryosurgery, freezing of malignant tissue is achieved with either single or multiple fine surgical probes which can be cooled to extremely low temperatures (less than minus one-hundred twenty degrees Celsius (−120° C.). The probes are inserted to the tissue with the guidance of imaging techniques like ultrasound. After the insertion, the probes are cooled. Ice balls form and grow from the surface of the cooled probes. Due to its minimally invasive characteristics and recent advances in monitoring technology during a surgery, cryosurgery is emerging as a promising treatment modality for prostate, liver and breast cancers. However, the understanding and precise control of the mechanism of freezing injury needs to be addressed for improved treatment efficacy.

Understanding the mechanism of freezing induced cell injury is an area of investigation in the field of cryobiology as it pertains to the applications of cryosurgery. A two-factor hypothesis was suggested to explain direct cell injury on the basis of two distinct freezing injury mechanisms—intracellular ice formation (IIF) and dehydration dominate the injury processes during freezing depending on the cooling rate of systems. When the cooling rate is rapid, cellular water nucleates and forms lethal intracellular ice. Otherwise, ice forms in the extracellular solution first and it leads to increased concentration of the unfrozen fraction. The increased extracellular concentration induces consequent cellular dehydration due to osmotic pressure difference. If this dehydration is too severe, then a form of toxicity or injury due to the high concentration of electrolytes can injure cells by mechanisms collectively called “solute” effects. IIF is generally considered to be lethal and believed to be the major injury mechanism at rapid cooling rates. However, solute effects injury appears more complex and is not fully understood yet.

One of the most substantial challenges in cryosurgical technique is due to incomplete tumor destruction near the ice ball edge, where tissues are frozen but not completely destroyed. The incomplete killing zone results in three potential problems. First, the freezing zone is typically larger than the size of tumor so as to ensure complete tumor destruction (i.e. surgical margin). This practice, however, can cause additional problems like healthy normal tissue destruction around a tumor, and sometimes is impractical where adjacent tissues, nerve system and/or organs need to be protected from freezing injury. Second, there is the possibility of recurrence of tumor after surgery due to incomplete destruction. The recurrence of tumor after surgery should be avoided. Finally, there is a limitation on the ability to monitor the complete killing zone during cryosurgery, as most available techniques keep track of the ice ball edge rather than the complete killing zone. Therefore, complete destruction of a given size of tumor can only be achieved using a surgical margin determined by surgeons' experience. Thus, there is a need in the art for improvement in the delivery and use of cryosurgical technique.

### SUMMARY OF THE INVENTION

The present invention provides a system and method of destroying and/or critically injuring tissue during cryosurgery, which is based on eutectic solidification within a biological system. This tissue destruction is believed to be the result, at least in part, of a direct cell injury mechanism caused by mechanical damage to the cells' membranes resulting from the eutectic solidification. In addition, the use of the present invention may also improve cryosurgery monitoring by bringing the edges of the killing zone and the ice ball closer together, thus providing surgeons with more complete injury information.

The present invention provides a composition, a method and/or a system of using the composition in cryosurgical destruction. In one embodiment, the composition includes one or more solutes that can effectively change a eutectic freezing point of biological materials. Biological materials can include, but are not limited to, cells, tumor cells, tissue, tumor tissues, tissues of internal organs such as liver tissue, prostate tissues, breast tissue, kidney tissues, and their associated fluids. In addition, biological materials can also include, but are not limited to vascular tissues, muscle tissues, including myocardium, tissues of the skin, connective tissues, and their associated fluid. Combinations of these biological materials are also possible.

The present invention can be useful in the treatment of, but not limited to, various cancers/tumors such as prostate cancer, liver cancer, breast cancer, uterine fibroids as well as any other tumor or tissue where cryosurgery has traditionally been used or might be used in future. Treatment of other physical conditions can also be possible.

In an additional embodiment, the present invention provides a composition, a method and/or a system for use in cryosurgery that allows for changing a eutectic freezing point of a biological material (e.g., a tissue). In one embodiment, the biological material (e.g., tissues and/or cells) to undergo cryosurgery may be identified, where at least a portion of the biological material is to undergo eutectic freezing. A eutectic changing composition may be introduced into the biological material, where the biological material can be treated with the composition for a time and an amount effective to change the eutectic freezing point and/or extend/strengthen the extent of eutectic solidification of the biological material. Therefore, the present invention may change the eutectic freezing point and/or extend or strengthen the extent of eutectic solidification (e.g., allow for a more complete eutectic solidification) within the biological material.

In one example, the composition for changing the eutectic freezing temperature is introduced into the portion of the biological material where eutectic freezing is desired. Introduction of the composition may be localized to the biological material mass for which eutectic freezing is desired. Alternatively, the composition may be localized to one or more portions of a biological material mass for which eutectic freezing is desired. Electronic visualization of the location of the composition in the biological material may be accomplished through the use of, e.g., contrast agents added to the composition for which electronic sensor can be used to electronically visualize the location of the composition. In one embodiment, the contrast agents may be visualized through any number of techniques, including, but not limited to ultrasound. Other visualization techniques may also be possible. For example, visualization might be achieved through the use of hypaque with fluoroscopy, gadalinium with MRI, impedance techniques (e.g., see U.S. Pat. No. 4,252,130 to Le Pivert), or possibly other methods.

The biological material treated with the eutectic changing composition may be cooled at a cooling rate that is effective to cause a eutectic formation in at least a portion of the biological material treated with the eutectic changing composition. In contrast to the biological material treated with the eutectic changing composition, biological materials not treated with the eutectic changing composition (e.g., tissues surrounding the treated tissues) may be less likely to undergo eutectic freezing. Thus, the eutectic changing composition may facilitate achieving a eutectic freeze primarily in the biological materials treated with the eutectic changing composition as compared to biological materials not so treated.

In one embodiment, the eutectic changing composition for use in a localized area of a mammal comprising at least one solute effective to change a biological material eutectic freezing point at the localized area of the mammal. For example, the eutectic changing composition may include at least one solute having a eutectic freezing temperature (when in solution) of no less than that of sodium chloride at a eutectic concentration, where the at least one solute is effective to change a biological material's eutectic freezing point. The at least one solute may be dissolved in a pharmaceutically acceptable solvent in an amount no greater than the eutectic concentration of the at least one solute.

In some aspects, the present invention may involve the use of a eutectic changing composition for the manufacture of a medicament for the treatment of biological materials.

The above summary of the present invention is not intended to describe each embodiment or every implementation of the present invention. Advantages, together with a more complete understanding of the invention, may become apparent and appreciated by referring to the following detailed description taken in conjunction with the accompanying drawings.

### BRIEF DESCRIPTIONS OF DRAWINGS

FIG. 1 provides a schematic illustration of a segment of tissue that is to undergo cryosurgical destruction according to one embodiment of the present invention.

FIG. 2 provides one example of a relationship of temperature versus distance from the ice ball center according to one embodiment of the present invention.

FIGS. 3A and 3B provide schematic illustrations of an ice ball formed during cryosurgery in which biological material has not been treated (FIG. 3A) or has been treated (FIG. 3B) with the eutectic changing composition of the present invention.

FIG. 4 provides one example of post-thaw viability changes of AT-1 cell suspensions in the 2×NaCl-water solution due to the presence of the eutectic solidification during a freezing/thawing protocol.

FIG. 5 provides one example of post-thaw viability changes of AT-1 cells in examples of eutectic changing compositions according to the present invention.

FIG. 6 provides DSC thermograms of rat liver tissues either treated with or not treated with a eutectic changing composition of the present invention. The solid line (--------) represents data of AT-1 tumor not tissue treated with a eutectic changing composition. The dashed line (- - - - -) represents data of AT-1 tumor tissue treated with a eutectic changing composition of potassium chloride (KCl). The linked line (--- - --- - ---) represents data of AT-1 tumor tissue treated with a eutectic changing composition of sodium chloride (NaCl).

FIGS. 7A-7F provide images of In vitro histology of AT-1 tumor tissues having undergone freeze/thaw experiments (400× magnification), where FIG. 7A shows control tissue, FIG. 7B shows freezing of tissue to −20° C., FIG. 7C shows tissue infused with KNO3 without freezing, FIG. 7D shows tissue infused with KNO₃ with freezing to −20° C., FIG. 7E shows tissue infused with KCl without freezing, and FIG. 7F shows tissue infused with KCl with freezing to −20° C.

FIGS. 8A and 8B show examples of DSC thermograms of rat liver tissues with/without infusing a half eutectic concentration KNO₃ solution according to one embodiment of the present invention.

### DETAILED DESCRIPTION OF ILLUSTRATIVE EMBODIMENTS

In the following detailed description of illustrative embodiments, reference is made to drawings that form a part hereof, and in which are shown by way of illustration specific embodiments in which the invention may be practiced. It is to be understood that other embodiments may be utilized and processing steps/structural changes may be made without departing from the scope of the present invention.

As will be discussed below, the present invention provides methods, compositions, and systems for changing a eutectic freezing point of biological materials (e.g., cells and tissues). As used herein, biological materials can include, but are not limited to cells and tissue that include cells, extracellular matrix structures (e.g., collagen, proteins), and associated biological fluids. In addition, the terms cells and/or tissues are used herein where it would be possible to also include, and/or exchange these terms for, any one of biological material, cells, and/or tissue.

Changes in the eutectic freezing point of the biological materials may provide for an enhancement of the destruction of the treated biological material in cryosurgery. As used herein, destruction and/or cryosurgical destruction can include the killing of cells and tissues of the biological material as a result of a cryosurgical procedure in which the present invention is used. The killing of the cells and tissues of the biological material may take place during any portion of the cryosurgical procedure, including the time after the completion of the cryosurgical procedure.

In addition, the present invention may provide for better assessment of the actual location of cell and tissue death in the ice ball formed during cryosurgery on the biological material. The present invention may also provide changes in the eutectic freezing point that allow for a greater percentage of cell and tissue destruction of the biological material during cryosurgery destruction.

Cryosurgical destruction has been shown to be an effective treatment modality for a variety of tumor tissues. In a surgical procedure for elimination of malignant tissue, it is important to take a sufficient surgical margin around the malignant tissue to ensure that all tumor tissue has been removed or destroyed. A sufficient margin using known techniques typically requires freezing beyond the tumor into normal tissue. To minimize the potential side effects of normal tissue damage during cryosurgery, and to maximize the tumor destruction at the edge of the cryosurgical ice ball, the present invention provides strategies to both protect (e.g., normal) and sensitize (e.g., tumor) cells to freezing are desirable.

The present invention may be used to destroy biological material of interest (e.g., tumors) and at the same time protect normal and healthy tissues around the biological material of interest near the ice ball edge where experienced temperature is between the ice formation temperature and the eutectic solidification temperature. As a result, it may be possible to minimize the surgical margin while decreasing damage to surrounding normal and healthy tissues. Increasing the efficiency of cell destruction near the ice ball edge might increase the confidence that an increased number of tumor cells were killed near the periphery of the biological material of interest while decreasing the chances of over-freeze damage into adjacent structure (such as the rectum in prostate cryosurgery). As used herein, the ice ball edge can be defined as the leading edge, of the ice formed by the cryosurgical probe.

The present invention may be used to more effectively destroy cells and tissue during cryosurgery based, in part, on a cell injury mechanism of eutectic solidification. In the field of cryobiology, two distinct cell injury mechanisms have been suggested. The first is the result of a solute effects injury that occurs during slow cooling rates. The second is the result of intracellular ice formation at rapid cooling rates. The present invention introduces an additional cell injury mechanism of a eutectic formation of eutectic crystallization. The eutectic formation of eutectic crystallization in cells and tissues for cryosurgery had not, up until this point, been recognized or thought to be a possible mechanism for cell injury and death. The present invention, however, recognizes that the formation of a eutectic freeze, and the formation of a eutectic crystallization, in cells and/or tissue system undergoing cryosurgery may enhance the destruction of the cells and/or tissue system.

The present invention provides for potential enhancement of cell and tissue destruction in cryosurgery by the use of eutectic freezing with the formation of eutectic crystallization. As used herein, eutectic formation and/or crystallization is defined as a solidification process through which water and solutes form a hydrate that can be recognized by a secondary heat release in differential scanning calorimetry (DSC).

One aspect of the present invention involves the use of a eutectic changing composition. Most physiological solutions are mixtures having water and physiologically acceptable electrolytes. Sodium chloride is one example of an acceptable electrolyte. Contrary to freezing of pure water, freezing physiological solutions typically results in at least two distinct thermal events. The first is the freezing of pure water in the solution. This occurs as the temperature of the solution falls below the freezing point of water, where the freezing point of the water is typically depressed due to the presence of the sodium chloride.

As the temperature continues to fall, more ice is formed. As the ice is formed, water is removed from the solution. As this happens, the sodium chloride concentration in the solution increases. As the temperature continues to fall, the eutectic point of the solution is reached at, e.g., approximately −21.1° C. For sodium chloride, the eutectic concentration (EuC) is 23.6% (wt./wt. NaCl to water), and at −21.1° C. crystals of both NaCl and the remaining water will form in the solution (eutectic solidification). With the formation of NaCl and water crystals, the eutectic point for sodium chloride has been reached.

As will be recognized, thermodynamic equilibrium is necessary in achieving the eutectic point freezing of −21.1° C. for sodium chloride. Typically, however, the eutectic freezing for a sodium chloride solution can be significantly delayed, i.e. supercooled, such as 40° C. or below. As used herein, supercooling includes the temperature difference between the thermodynamic equilibrium eutectic temperature and the actual temperature where the eutectic solidification occurs. Each salt has its own eutectic temperature and concentration that may be the same or different than those of sodium chloride.

Eutectic formation may cause significant direct cell injury at slow cooling rates, especially when the temperature at which the eutectic formation occurs can be enhanced (e.g., the eutectic temperature of the biological material can be increased). Slow cooling rates can include, for example, those having a cooling rate of 1° C./minute or greater (i.e., no less than 1° C./minute). Alternatively, the slow cooling rates can include those having a cooling rate of 1° C./minute to 10° C./minute. It is understood, however, that eutectic solidification can occur at many different cooling rates, including those no less than 10° C./minute or and/or those no greater than 1° C./minute. Enhancing the eutectic formation may include changing the micro-environment of the cells and/or tissues so as to increase and/or create a eutectic freezing point of the biological material that can be achieved during a cryosurgical procedure.

When cells suspended in physiological solutions are frozen at slow cooling rates, ice crystals are typically formed in the extracellular space. As the temperature falls, the ice crystals grow and the concentration of the unfrozen fraction of solution increases. Meanwhile, cells are suspended in the highly concentrated unfrozen fraction among ice crystals. As the temperature continues to drop, eutectic formation is induced in the unfrozen fraction and directly damages cells in the unfrozen fraction by simultaneous solidification in a new solid phase referred to as the eutectic solidification.

As described above, eutectic solidification may cause significant direct cell injuries at slow cooling rates. Controlling the point at which the eutectic solidification occurs in cells and/or tissue of the biological material can significantly influence the degree to which the cryosurgical destruction will be successful. Using solutes whose eutectic freezing temperatures when in solution are no less than that of sodium chloride at its eutectic concentration allows for beneficial changes in the tissue eutectic freezing point.

In one example, the at least one solute used to change the eutectic freezing temperatures are in a pharmaceutically acceptable solvent in which the at least one solute can be dissolved in an amount no greater than the eutectic concentration of the at least one solute. In other words, the solute used to change the eutectic freezing point or extend/strengthen the extent of eutectic solidification is used at a concentration at or below (i.e., no greater than) is eutectic concentration (wt./wt.) value. A pharmaceutically acceptable solvent can include, but is not limited to, water, where the water could have been distilled (e.g., double distilled), deionized, and/or sterilized (e.g., filter purified and/or heat and pressure sterilized), using techniques as are known.

Useful solutes for changing the temperature of a eutectic formation may include, but are not limited to, the following:

| | | % Eutectic |
|---|---|---|
| Solute | Eutectic Temp. (° C.) | Concentration (wt./wt.) |
| KNO₃ | −2.9 | 10.9% |
| KCL | −11.1 | 19.7% |
| MgSO₄ | −3.9 | 19.0% |
| NaCl | −21.8 | 23.6% |
| KBr | −13.0 | — |
| NH₄Cl | −15.8 | 18.6 |
| MgCl₂ | −33.6 | 21.6 |
| CaCl₂ | −55 | 29.8 |
| Glucose | −5.0 | 32.0 |
| Sucrose | −13.5 | 62.5 |

In one embodiment, a hypertonic NaCl solution may be used to change the eutectic point of cells and/or tissue of biological material. Alternatively, infusing concentrated solutes whose eutectic freezing temperatures are higher than that of NaCl can change the eutectic freezing point or extend/strengthen the extent of eutectic solidification. Solutions having two or more solutes (i.e., two or more salts) are also possible, where the resulting eutectic temperature and concentration of the solution can be different than any of the two or more solutes alone.

These methods can improve cryosurgery protocols by providing a controllable and reproducible technique to accentuate mechanistic freezing injury (i.e., eutectic formation in and around cells) of malignant cells and tissues.

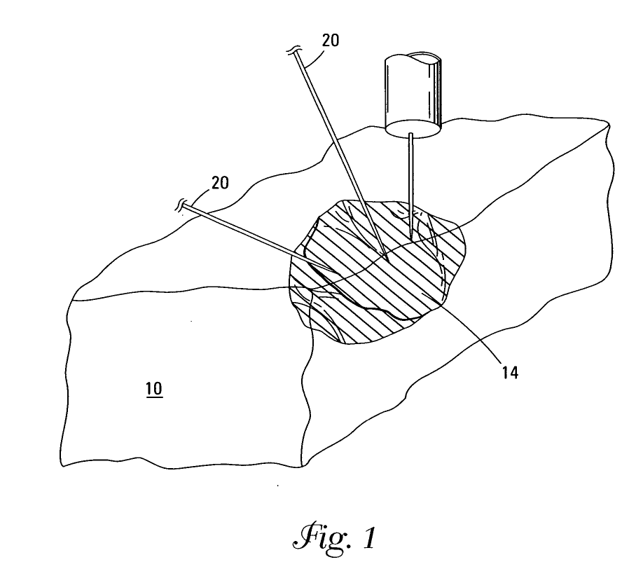

FIG. 1 shows one embodiment of a segment of tissue 10 that includes a portion 14 that is to undergo cryosurgical destruction. The portion 14 of tissue 10 can have a similar cell and/or tissue structure as the surrounding segment of tissue 10. Alternatively, the portion 14 can have one or more morphologically distinct cell and/or tissue structures as compared to the remaining segment of tissue 10. In one example, the portion 14 can be a tumor.

The eutectic freezing point of the portion 14 of the tissue may be changed relative to the remaining segment of tissue 10 through the use of the eutectic changing composition of the present invention. The portion 14 of the tissue 10 to undergo eutectic freezing during cryosurgical destruction may be identified by any number of known techniques. For example, tumor structures may be identified through tissue structure, biological markers, ultrasound, or any number of other techniques.

The portion 14 of tissue to undergo eutectic freezing may then be treated with a eutectic changing composition for a time, an amount, and a type effective to change the eutectic freezing point or extend/strengthen the extent of eutectic solidification of the portion 14 of the tissue. In one example, the eutectic changing composition can include one or more of the solutes for changing the temperature of a eutectic formation as discussed herein. In addition, the solutes of the eutectic changing composition can be provided at their eutectic concentration, or any effective fraction, or percentage, thereof.

In one example, the eutectic changing composition can be injected into one or more locations of the portion 14 of the tissue. U.S. Pat. No. 5,807,395 provides some examples of catheters suitable for injecting the eutectic changing composition of the present invention. In addition, the eutectic changing composition can be introduced into the one or more locations through, e.g., the use of hypodermic needles, one or more needles attached to a cryoprobe, diffusion, and/or iontophoresis (or any other use of electric fields to drive ionic solution flow in tissues).

The location and/or extent to which the eutectic changing composition has been infused into the tissue (e.g., the portion 14 in FIG. 1) can be monitored through any number of techniques. For example, compounds and/or solutions that may enhance ultrasonic imaging, fluoroscope, MRI, impedance technique (e.g., U.S. Pat. No. 4,252,130 to Le Pivert) can be added to the eutectic changing composition to allow for visualization of the location of the eutectic changing composition. Examples include, but are not limited to contrast agent added with salt (i.e., hypaque), salt tagged with a fluorescent marker, and/or use of an impedance metric device to see how impedance changes locally with infusion.

One or more cooling probes 20 are then used to cool the portion 14 of the tissue 10 at a cooling rate effective to cause a eutectic formation in at least the portion 14 of the tissue 10.

During cooling of the tissue, an ice ball is preferably formed. The ice ball formation typically originates at or about the tip of each cooling probe. As the cooling probe, or probes, removes heat from the tissue, the ice ball grows. Visualizing the perimeter of the ice ball formation can be an important factor in determining the extent, or amount, of tissue and cell material that are killed during the cryosurgical procedure. Visualization of the perimeter of the ice ball may be accomplished, e.g., through the use of ultrasonic imaging.

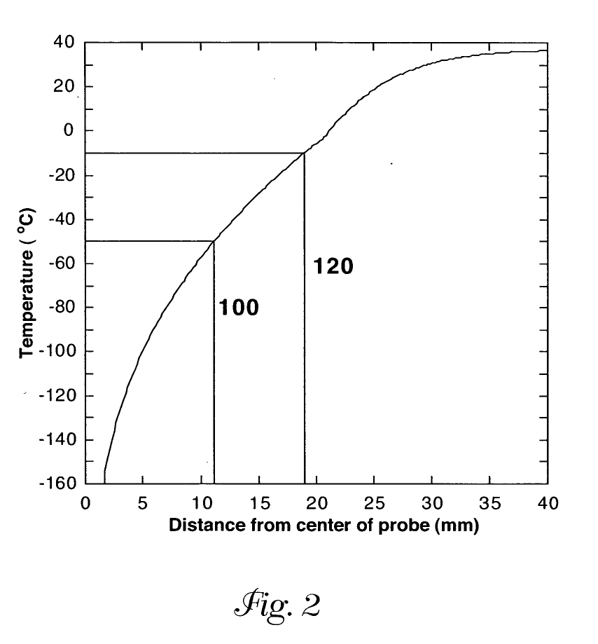

FIG. 2 depicts one example of the relationship of temperature versus distance from the ice ball center. Line 100 illustrates the distance from the center of the ice ball (e.g., the location of the cooling probe) where cell death will typically occur for tissue that has not been treated with the eutectic changing composition. As will be noted, the temperature at the distance where the cell death is suggested to occur within tumors is approximately minus fifty (−50) degrees Celsius (C°) in the depicted example. In contrast, when the tissue is treated with the eutectic changing composition as described herein, the distance from the center of the ice ball (e.g., the location of the cooling probe) where cell death will typically occur is increased along with the temperature. This is illustrated by line 120. Thus, the eutectic changing composition may effectively increase the distance from the cooling probe for which cell death will typically occur.

In addition to increasing the distance from the cooling probe where cell death will typically occur, the use of the eutectic changing composition may also change the size, and/or extent, of the perimeter of the ice ball. For example, the use of the eutectic changing composition may reduce the perimeter of the ice ball as compared to same conditions without the use of the eutectic changing composition. Although not wishing to be bound by theory, it is thought that this is due, in part, to the effect of a freezing point depression caused by the introduction of the eutectic changing composition. The reduction in the perimeter of the ice ball formation coupled with the increase at which cell death will typically occur in the ice ball results in an ice ball with a perimeter that more closely defines where the actual cell death occurs, or will occur.

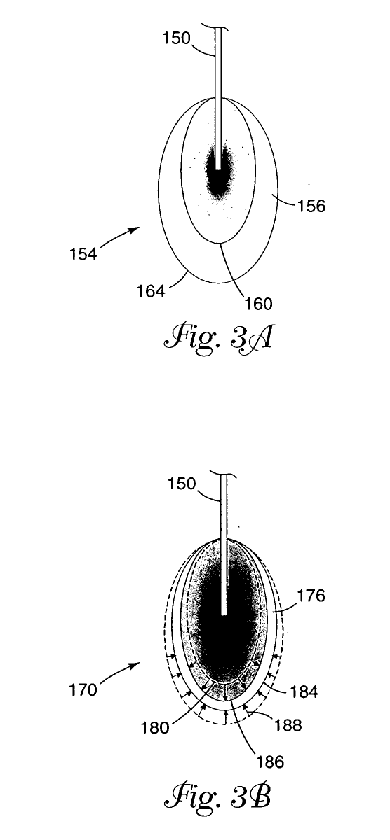

FIGS. 3A and 3B illustrate this latter point. FIG. 3A illustrates cryosurgical freezing probe 150 positioned in biological material 154. Cryosurgical freezing probe 150 is used to remove heat from the biological material 154 so as to form ice ball 156. The ice ball 156 typically includes at least a first volume 160 and a second volume 164 of the biological material 154. The first volume 160 of the biological material 154 is typically in closer proximity to the cryosurgical freezing probe 150 than the second volume 164 of biological material 154. The first volume 160 of the ice ball 156 typically defines a volume of the biological material 154 that is essentially destroyed during the cryosurgical procedure. This first volume 160 of tissue can be referred to as a killing zone during the cryosurgical procedure. The second volume 164 of the ice ball 156 typically defines a volume of the biological material 154 surrounding the first volume 160 that is either partially or not destroyed, but may undergo freezing, or at least partial freezing, during the cryosurgical procedure. This second volume 164 of tissue can be referred to as an incomplete killing zone during the cryosurgical procedure.

The presence of this second volume 164 of tissue (the incomplete killing zone) can result in at least three potential problems. First, a freezing zone larger than the size of tumor may be required to ensure complete tumor destruction (i.e. surgical margin). Second, there remains the possibility of recurrence of, for example, a tumor after surgery due to its incomplete destruction. Finally, there can be a limitation on the ability to monitor the complete first volume 160 (the killing zone) of the biological material 154 during cryosurgery.

The above potential problems can be lessened by use of the eutectic changing composition of the present invention during cryosurgery. If the biological material is first treated with the eutectic changing composition according to the present invention, the killing zone of the first volume 160 of biological material may be enlarged (enlarged kill zone), while the second volume 164 of the ice ball 156 is reduced (smaller incomplete kill zone), relative to biological material not treated the eutectic changing composition of the present invention.

FIG. 3B provides an example of this latter point. In FIG. 3B, biological material 170 has been treated with a eutectic changing composition according to the present invention. Cryosurgical freezing probe 150 can be positioned in biological material 170 and used to remove heat from the biological material 170 so as to form a eutectic enhanced ice ball 176. The eutectic enhanced ice ball 176 typically includes at least a first volume 180 and a second volume 184 of the biological material 170. The first volume 180 of the biological material 170 is typically in closer proximity to the cryosurgical freezing probe 150 than the second volume 164 of biological material 170. The first volume 180 of the ice ball 176 typically defines a volume of the biological material 170 that is essentially destroyed during the cryosurgical procedure (i.e., the killing zone). The second volume 184 of the eutectic enhanced ice ball 176 typically defines a volume of the biological material 170 surrounding the first volume 180 that is either partially or not destroyed, but may undergo freezing, or at least partial freezing, during the cryosurgical procedure (i.e., the incomplete killing zone).

Comparing the portions of the ice balls shown in FIGS. 3A and 3B illustrates at least one effect of the cryosurgical composition of the present invention for comparable cryosurgical procedures (e.g., comparable freezing rates). As shown in FIG. 3B, the first volume 180 of the eutectic enhanced ice ball 176 has been enlarged relative the first volume 160 of ice ball 156 (FIG. 3A). This enlargement of the volume of the “killing zone” relative to the first volume 160 of ice ball 156 in the untreated biological material 154 is shown a 186 in FIG. 3B. It is believed that this enlargement 186 of the first volume 180 is due to the use of the cryosurgical composition of the present invention.

In addition to increasing the “killing zone” in the eutectic enhanced ice ball 176, the use of the cryosurgical composition of the present invention is also believed to decrease the overall volume of the eutectic enhanced ice ball 176 (e.g., perimeter of eutectic enhanced ice ball 176 reduced) relative to the volume of ice ball 156 in the untreated biological material 154. This reduction in the volume of the eutectic enhanced ice ball 176 relative to the volume of ice ball 156 is shown at 188 in FIG. 3B. The reduction in the volume of the eutectic enhanced ice ball 176 relative to the volume of ice ball 156 in the untreated biological material 154 is believed to be the result of a freezing point depression resulting from the use of the cryosurgical composition of the present invention.

As discussed herein, the reduction in the perimeter of the ice ball and the increase in the kill zone are both due to use of the eutectic changing composition of the present invention. One potential beneficial result of this change in the first volume 180 and the overall volume of the eutectic enhanced ice ball 176 relative to the volumes of ice ball 156 in the untreated biological material 154 is that the killing zone of the first volume 180 may more closely correlate with the perimeter of the second volume 184 of the eutectic enhanced ice ball 176. This may allow a more accurate prediction of the actual killing zone created during the cryosurgical destruction procedure.

As discussed above, the present invention may also provide a composition, method and/or system of using the composition described herein in cryosurgical destruction. The composition may include one or more solutes that can effectively change eutectic freezing point of the biological materials exposed to the eutectic changing composition. As discussed, the eutectic changing composition may include a composition for use in a localized area of any native or artificial tissue of a mammal comprising, as an active ingredient, at least one solute effective to change the tissue eutectic freezing point at the localized area of the native or artificial tissue of the mammal.

In one embodiment, the system of the present invention may include the eutectic changing composition, as described herein, dissolved in a pharmaceutically acceptable solvent, and a catheter having a lumen, where the eutectic changing composition can move through the lumen of the catheter and into the tissue for which a change in a eutectic freezing point is desired. The catheter of the present invention may also include a needle at a distal end of the catheter for delivering the eutectic changing composition. Alternatively, the catheter can further include a trocar in the lumen of the catheter to facilitate delivering a portion of the catheter to the tissue for which a change in a eutectic freezing point is desired.

As discussed, U.S. Pat. No. 5,807,395 provides some examples of catheters suitable for injecting the eutectic changing composition of the present invention. The system may also include at least one probe, where the probe can remove thermal energy from the location for cryosurgical destruction at a rate, as discussed herein, sufficient to cause tissue at the location for cryosurgical destruction to undergo eutectic freezing.

It may also be possible to include additional additives with the solutes with the eutectic changing composition. For example, additional additives might include, but are not limited to, a composition to further enhance cell and tissue destruction by cryosurgery. U.S. Pat. No. 5,654,279 to Rubinsky et al. provides one example of possible additional additives. In addition chemotherapeutic agents can also be introduced with the eutectic changing composition.

Objects and advantages of the present invention are further illustrated by the following examples, but the particular materials and amounts thereof recited in these examples, as well as other conditions and details, should not be construed to unduly limit this invention.

### EXAMPLES

The present examples provide an illustration of the use of the eutectic changing composition of the present invention in eutectic formation within biological materials for destroying malignant tissue during cryosurgery. Generally, the eutectic crystallization was induced by infusing a eutectic changing composition of the present invention into AT-1 rat prostate tumor (cell suspensions/tissues) and normal rat liver tissues. Post-cryosurgery viability of AT-1 cell suspensions in various media was also assessed at temperatures above and below eutectic formation. Inducing eutectic crystallization in tissues during freezing was done with normal rat liver and AT-1 tumor tissues, and the corresponding freezing injury enhancement was assessed after a freeze/thaw. The results provide biophysical evidence of the eutectic induced freezing injury in tissue and may lead to improvement in the delivery and use of cryosurgical technique.

### Example 1

AT-1 rat prostate tumor cells were used in the following example. The AT-1 rat prostate tumor cells were cultured in vitro under standard tissue culture conditions, as are known. Cultured AT-1 cells were separated from a culture flask by immersion in 0.05% (by volume) trypsin and 0.53 mM EDTA, and then suspended in 5% (by volume) fetal bovine serum (FBS)-supplemented medium such that the final trypsin concentration was <0.005% (by volume). After the separation, the cells were pelleted by centrifugation and the excess medium was removed. The cell pellet was re-suspended into various aqueous solutions (about 1.0 ml) before experiments and nominal cell concentration was about 2×10⁶ cells/mi. The suspensions were stored in 1.5 ml microcentrifuge tube on ice (about 4° C.).

To investigate biophysical phenomena during freeze/thaw, a DSC (Pyris 1, Perkin-Elmer Corporation, Norwalk, Conn.) was used. The temperature scale of the DSC was calibrated with two different transition temperatures of cyclohexane (−85.8° C. and 6.4° C.). The heat flow scale of the DSC was calibrated against the heat of fusion of pure water (335 J/g) during thawing at 5° C./min.

A directional solidification stage consisting of two constant temperature reservoirs that are held at different temperature and separated by an adjustable gap was used in the experiments. The first reservoir was held at suprazero temperature (above 0° C.) and the second reservoir at subzero temperature (below 0° C.). The sample rested in a 3 mm wide and 1 mm deep well on a microscope microslide. The glass microslide was moved from the first reservoir (suprazero temperature) to the second reservoir (subzero temperature) over the gap at a precisely controlled velocity. By appropriately setting the microslide velocity, gap size, and reservoir temperatures, constant cooling rates and precise end temperatures can be imposed on the cell suspension.

Controlled cooling and thawing rate were achieved by the DSC and directional solidification stage. Unless otherwise mentioned, cooling and thawing rates were 5° C./min.

For the directional solidification stage, fast thawing rates (about 200° C./min) were employed. To obtain a rapid thawing rate, the glass microslide was removed from the directional solidification stage and quickly placed on an aluminum block at 37° C.

Post-thaw viabilities of AT-1 cell suspensions in various media were assessed for varying end temperature of the freezing and thawing protocol on the directional solidification stage. Viability of the AT-1 cell suspensions was measured by a membrane integrity assay using Hoechst and Propidium Iodide. About 10 μl samples were collected after the freeze/thaw protocol and incubated with 0.01 μl Hoechst and 0.01 μl Propidium Iodide for 15 minutes at 37° C. After incubation, viability was assessed under a fluorescent microscope by scoring more than 200 cells for each sample.

AT-1 cells were suspended in a 2×NaCl-water solution. The suspended AT-1 cells underwent a freezing and thawing protocol on the cryomicroscope stage. The detailed protocol consisted of i) freezing from room temperature (about 20° C.) to −25° C. at a cooling rate of 5° C./minute, ii) holding at −25° C. for 3 minutes; and iii) thawing to room temperature at a heating rate of 130° C./minute. The only difference in the protocol between two AT-1 cell suspension groups was that eutectic solidification was initiated in one group at the beginning of the holding step (step ii) by touching the edge of the samples with a pre-cooled needle, which had been submerged in liquid nitrogen. Three minute hold time was long enough for the eutectic crystallization to propagate across the entire sample. These freezing and thawing conditions were possible since the end temperature, −25° C., lay between the eutectic solidification temperature and the thermodynamic equilibrium eutectic temperature of NaCl-water. The occurrence or nonoccurrence of the eutectic crystallization were visually confirmed in each experimental group, since the eutectic crystallization can cause a distinct opacity change from transparent to opaque in the medium.

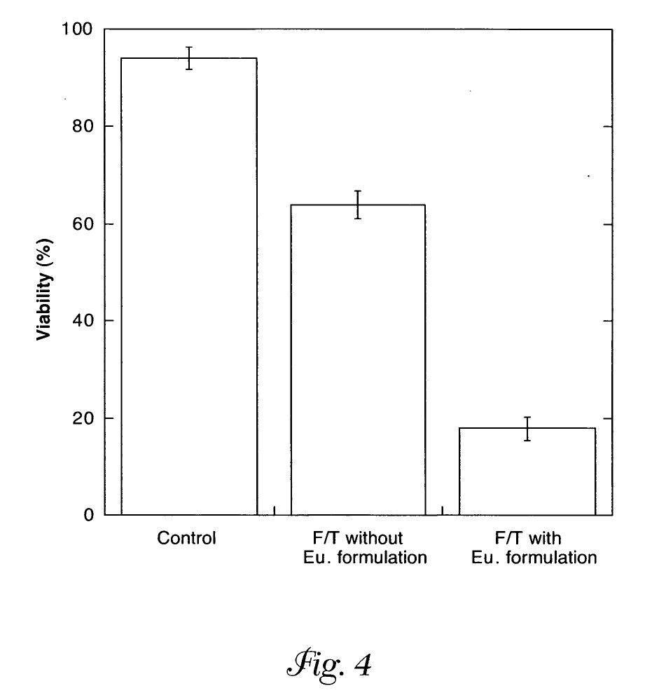

FIG. 4 shows the post-thaw viability changes of AT-1 cell suspensions in the 2×NaCl-water solution due to the presence of the eutectic solidification during a freezing/thawing protocol. The viability of the control AT-1 cell suspensions remained as high as 95% even when the AT-1 cells were suspended in the hypertonic saline. After the freezing/thawing protocol to −25° C., the viability of the AT-1 cells that did not undergo eutectic solidification decreased to about 64%. While not wishing to be bound by theory, this may have been due to a tradition form of solute effects injury by high electrolyte concentration. When the eutectic formation occurs in the samples during the same freeze/thaw freezing/thawing protocol to −25° C., the viability decreased to about 17%. In this case, the eutectic solidification decreased viability by nearly 50% in an otherwise identical freezing/thawing protocol. Since both groups were frozen at the same end temperature through the same thermal history except the occurrence of the eutectic solidification, cell in both systems undergo the same elevated electrolyte concentration. This would suggest that the differences in viabilities are caused by injury associated with the occurrence of eutectic solidification.

### Example 2

To induce eutectic formation, potassium nitrate (KNO₃), potassium chloride (KCl) and sodium chloride (NaCl) were used in a eutectic changing composition based on their eutectic temperature and concentration as summarized in Table 1, below. The eutectic changing solutions for each of these salts were prepared at a half eutectic concentration (potassium nitrate solution is 5.4% wt./wt., potassium chloride solution 9.85%, and sodium chloride solution 11.8% wt./wt.). In freezing experiments with cell suspensions, a half eutectic concentration solution was mixed with cell culture media (Dulbecco's Modified Eagle's Medium/F-12) in 1 (salt solution): 2 (culture media) volume ratio.

AT-1 rat prostate tumor cells were cultured in vitro under standard tissue culture conditions, as are known. AT-1 cells were suspended in each eutectic changing solution, and kept at about 4° C. Viability changes after mixing in high concentration salt in controls were less than 5% for 2 hours.

TABLE 1
Salts used to induce eutectic crystallization during freezing

| | |
|---|---|
| | Eutectic |
| Eutectic Temperature | Concentration |

| Salts | (° C.) | (K) | (% wt./wt.) |
|---|---|---|---|
| KNO₃ | −2.9 | 270.3 | 10.9 |
| KCl | −11.1 | 262.1 | 19.7 |
| NaCl | −21.8 | 251.4 | 23.6 |

Cultured AT-1 cells were separated from a culture flask by immersion in 0.05% (by volume) trypsin and 0.53 mM EDTA, and then suspended in 5% (by volume) fetal bovine serum (FBS)-supplemented medium such that the final trypsin concentration was <0.005% (by volume). After the separation, the cells were pelleted by centrifugation and the excess medium was removed. The cell pellet was re-suspended into various aqueous solutions (about 1.0 ml) before experiments and nominal cell concentration was about 2×10⁶ cells/ml. The suspensions were stored in 1.5 ml microcentrifuge tube on ice (about 4° C.).

To investigate biophysical phenomena during freeze/thaw, a DSC (Pyris 1, Perkin-Elmer Corporation, Norwalk, Conn.) was used. The temperature scale of the DSC was calibrated with two different transition temperatures of cyclohexane (−85.8° C. and 6.4° C.). The heat flow scale of the DSC was calibrated against the heat of fusion of pure water (335 J/g) during thawing at 5° C./min.

A directional solidification stage consisting of two constant temperature reservoirs that are held at different temperature and separated by an adjustable gap was used in the experiments. The first reservoir was held at suprazero temperature (above 0° C.) and the second reservoir at subzero temperature (below 0° C.). The sample rested in a 3 mm wide and 1 mm deep well on a microscope microslide. The glass microslide was moved from the first reservoir (suprazero temperature) to the second reservoir (subzero temperature) over the gap at a precisely controlled velocity. By appropriately setting the microslide velocity, gap size, and reservoir temperatures, constant cooling rates and precise end temperatures can be imposed on the cell suspension.

Controlled cooling and thawing rate were achieved by the DSC and directional solidification stage. Unless otherwise mentioned, cooling and thawing rates were 5° C./min.

For the directional solidification stage, fast thawing rates (about 200° C./min) were employed. To obtain a rapid thawing rate, the glass microslide was removed from the directional solidification stage and quickly placed on an aluminum block at 37° C.

Post-thaw viabilities of AT-1 cell suspensions in various media were assessed for varying end temperature of the freezing and thawing protocol on the directional solidification stage. The results are shown in FIG. 4B. Briefly, the freezing and thawing protocol was i) freezing a sample from 4° C. to a given end temperature at 5° C./minute; and ii) thawing at 37° C. at about 200° C./minute. Control solutions used were an isotonic NaCl-water (1×NaCl-water) solution and the AT-1 culture medium.

To induce eutectic solidification, in the cell suspensions at different temperatures, the half eutectic concentrations of potassium nitrate (KNO₃) in water (KNO₃-water) or potassium chloride (KCl) in water (KCl-water) were mixed with AT-1 culture media at a 1:2 volume ratio (1 KNO₃-water or KCl-water solution: 2 AT-1 culture media). The concentrations of these solutions were determined so that the use of these solutions would not result in excessive killing of the AT-1 cells due to high osmotic pressure. The viability of control AT-1 cells in these test solutions was greater than 90% after an hour at about 4° C.

Viability of the AT-1 cell suspensions was measured by a membrane integrity assay using Hoechst and Propidium Iodide. About 10 μl samples were collected after the freeze/thaw protocol and incubated with 0.01 μl Hoechst and 0.01 μl Propidium Iodide for 15 minutes at 37° C. After incubation, viability was assessed under a fluorescent microscope by scoring more than 200 cells for each sample.

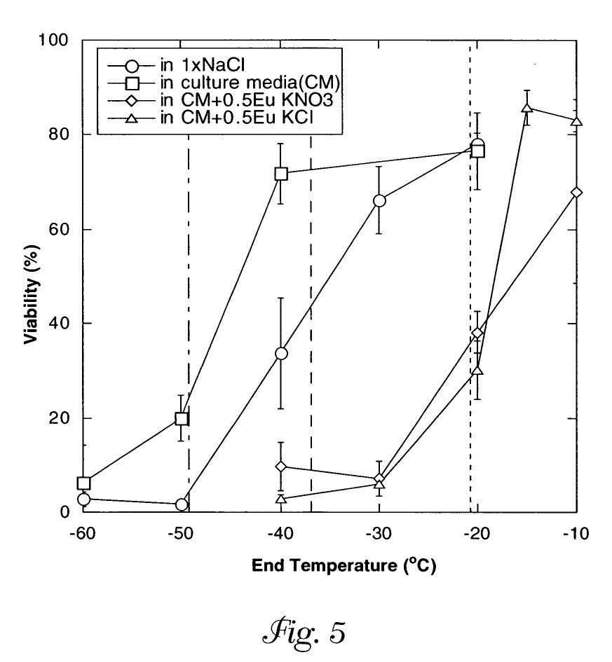

FIG. 5 shows the post-thaw viability of AT-1 cells suspensions in the media described above on the directional solidification stage. The onset temperature of the eutectic solidification was measured using the DSC, as described herein. Generally speaking, the viability of the AT-1 cells in the suspension solutions decreased with end temperature regardless of the media used. Note that the temperatures of noticeable viability drop to coincide with the eutectic crystallization temperatures of each suspension media. When the viability of the AT-1 cell suspension in the 1×NaCl (the onset of eutectic crystallization at about −37° C.) is compared with that of KCl infused AT-1 cell suspension (the onset of eutectic crystallization at about −21° C.), there is 70% less viability at −30° C. in the KCl infused AT-1 cell suspension.

A similar change was seen by comparing the viabilities of the AT-1 cell suspension in the culture medium and 1×NaCl-water at −40° C., where the viability in 1×NaCl-water is 50% lower than the viability in the culture medium. There was also a distinction between the viability of the potassium nitrate (KNO₃) infused AT-1 cell suspension and the potassium chloride (KCl) infused AT-1 cell suspension at about −10° C., where the AT-1 cell suspension in the potassium nitrate (KNO₃) infused medium experiences eutectic solidification, but the AT-1 cell suspension in the potassium chloride (KCl) infused medium does not. At this temperature (about −10° C.), the viability of the AT-1 cells in the potassium chloride (KCl) infused medium was 10 to 20 percent higher than in the potassium nitrate (KNO₃) infused medium. Based on traditional solute effects injury by elevated electrolyte concentrations, the viability of the potassium chloride (KCl) infused medium should be lower than in the infused potassium nitrate (KNO₃) infused medium. These results, therefore, indicate that the eutectic solidification was detrimental to cells during at least freezing. They also imply the possibility of enhancing direct cell injury during freezing and thawing by the addition of other solutes having higher eutectic temperatures to a eutectic changing composition.

### Example 3

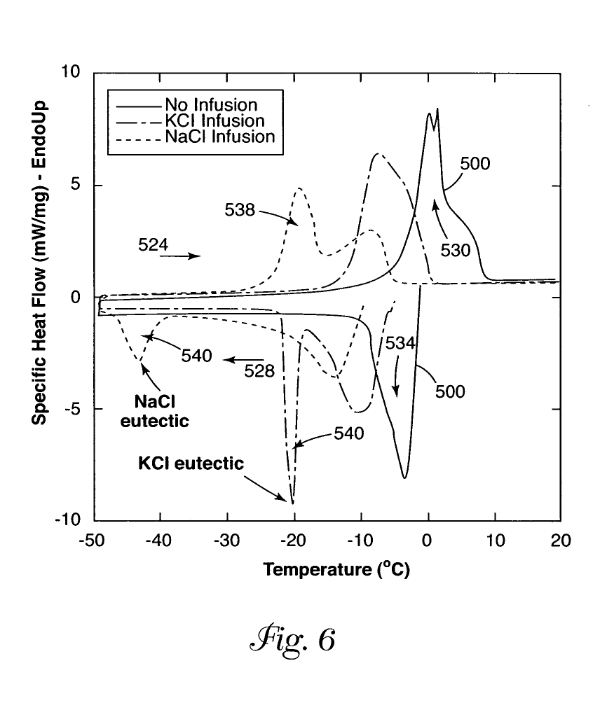

FIG. 6 shows DSC thermograms of rat liver tissues either treated with or not treated with a eutectic changing composition of the present invention. The solid line (------) 500 represents data of AT-1 tumor not tissue treated with a eutectic changing composition. The dashed line (- - - - -) 510 represents data of AT-1 tumor tissue treated with a eutectic changing composition of potassium chloride (KCl) at a half eutectic concentration, as described herein. The linked line (--- - --- -) 520 represents data of AT-1 tumor tissue treated with a eutectic changing composition of sodium chloride (NaCl) at a half eutectic concentration, as described herein. The tissues were isolated and underwent freezing 528 and heating 524 (i.e., thawing) as described above.

The tissue without infusion, line 500, had a heat absorption peak 530 and a heat release peak 534, which were associated with water/ice phase change. However, when the eutectic changing composition of the present invention were infused into the tissue, a secondary heat absorption peak 538 and a secondary heat release peak 540, both associated with eutectic phase change, were observed. Based on this information, it is believed that eutectic crystallization can be induced by infusion or diffusion of solutes of the eutectic changing composition into a biological material.

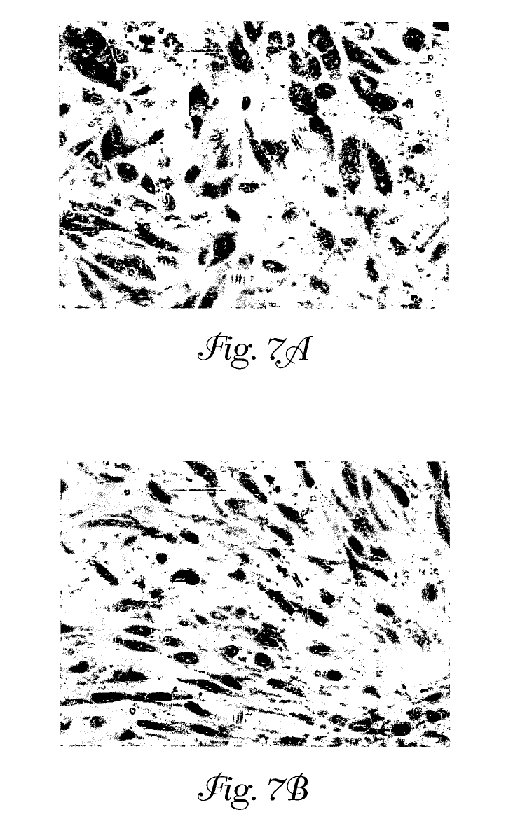

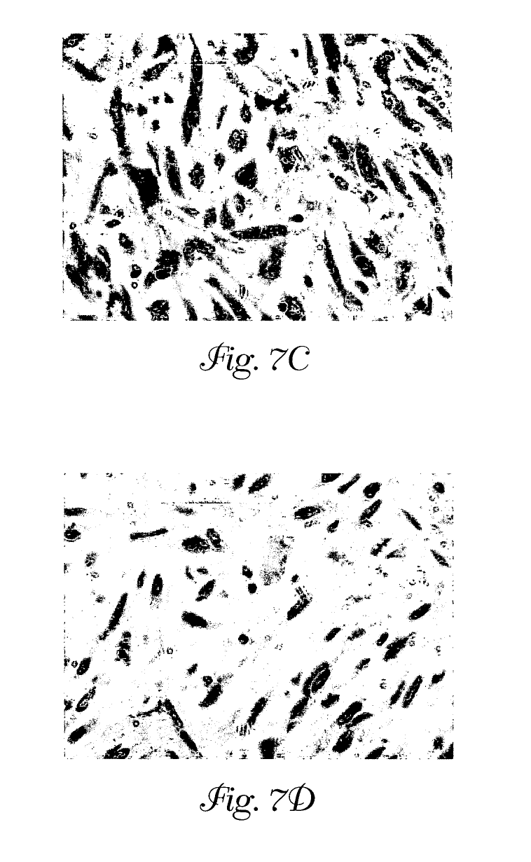

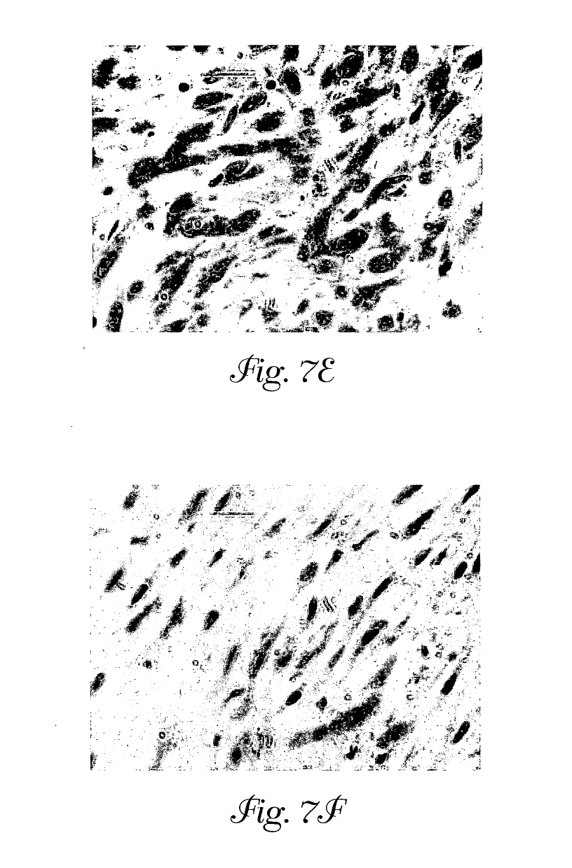

FIGS. 7A-7F show images of histology preparations of AT-1 tumor tissues 2 days after the freezing experiments on the directional solidification stage. Control tissues are very similar in all cases (FIGS. 7A, 7C, and 7E). Some cytoplasmic retraction is seen after salt infusion in FIGS. 7C and 7E over the control sample (FIG. 7A), but overall viability appears high. After freezing the samples to −20° C., a reduction in the number and quality of nuclei in all frozen samples is noted (FIGS. 7B, 7D, and 7F). Nuclear changes include darkening, reduction in size of chromatin, pykonsis and in some cases loss of nuclear material. In addition, cell membranes in frozen salt infused samples are indistinct and difficult to identify. All of these changes appear accentuated in the KNO₃ and KCl infused samples.

### Example 4

To induce eutectic formation, potassium nitrate (KNO₃) was used in a eutectic changing composition based on its eutectic temperature and concentration as summarized in Table 1, above. A solution of KNO₃ was prepared at a half eutectic concentration (potassium nitrate solution is 5.4% wt./wt.). In freezing experiments with cell suspensions, a half eutectic concentration solution was mixed with cell culture media (Dulbecco's Modified Eagle's Medium/F-12) in 1 (salt solution): 2 (culture media) volume ratio.

AT-1 cells were suspended in the solution, and kept at about 4° C. Viability changes after mixing in high concentration salt in controls were less than 5% for 2 hours. For tissue freezing experiments, each solution was infused into tissue slices by injection of the solution (about 50 to 100 μl) into the tissue samples using a hypodermic needle. After the infusion, excessive solution was removed with absorbent paper towels.

AT-1 rat prostate tumor cells were cultured in vitro, as described above. Cultured AT-1 cells were separated from a culture flask by immersion in 0.05% (by volume) trypsin and 0.53 mM EDTA, and then suspended in 5% (by volume) fetal bovine serum (FBS)-supplemented medium such that the final trypsin concentration was <0.005% (by volume). After the separation, the cells were pelleted by centrifugation and the excess medium was removed. The cell pellet was re-suspended into various aqueous solutions (about 1.0 ml) before experiments and nominal cell concentration was about 2×10⁶ cells/ml. The suspensions were stored in 1.5 ml microcentrifuge tube on ice (about 4° C.).

AT-1 tumors were seeded by subcutaneous injection of 2×10⁶ AT-1 cells in 100 μl of Hanks' balanced salt solution in the flank region of male Copenhagen rats (about 250 g) (Harlan-Spraque-Dawley, Inc., Indianapolis, Ind.). Tumors were grown to a size of 2-3 cm in the largest dimension, and harvested from the rats. Liver tissues were also isolated from the rats. After the harvest and isolation, the tissues were placed in a petri dish with culture media. Using a razor blade or a precision cutter, tissues were sliced in 3 mm long, 3 mm wide and 3 mm thick slice for freezing experiments.

To investigate biophysical phenomena during freeze/thaw, a DSC (Pyris 1, Perkin-Elmer Corporation, Norwalk, Conn.) was used. The temperature scale of the DSC was calibrated with two different transition temperatures of cyclohexane (−85.8° C. and 6.4° C.). The heat flow scale of the DSC was calibrated against the heat of fusion of pure water (335 J/g) during thawing at 5° C./min.

The directional solidification stage, as described above, was used in the experiments. Controlled cooling and thawing rate were achieved by the DSC and directional solidification stage. Unless otherwise mentioned, cooling and thawing rates were 50° C./min.

For the directional solidification stage, fast thawing rates (about 200° C./min) were employed. To obtain a rapid thawing rate, the glass microslide was removed from the directional solidification stage and quickly placed on an aluminum block at 37° C.

Post-thaw viabilities of AT-1 cell suspensions in various media were assessed. For tissue samples in cell culture media alone frozen to about −50° C., viability was 62.7±7.5%, with the control samples (no freeze/thaw procedure) having a viability of 98.6%. For tissue samples treated with the KNO₃ solution prepared at half eutectic concentration and frozen to about −20° C., the viability was 15.2±7.1%, with the control samples having a viability of 92.1%. Comparative data from Smith et al. (Smith, et al., “A parametric study of freezing injury in AT-1 rat prostate tumor cells”, Cryobiology 39, 13-28, 1999) indicates viability of AT-1 cell suspensions in culture media through the same freezing protocol were 74.7±4.6%.

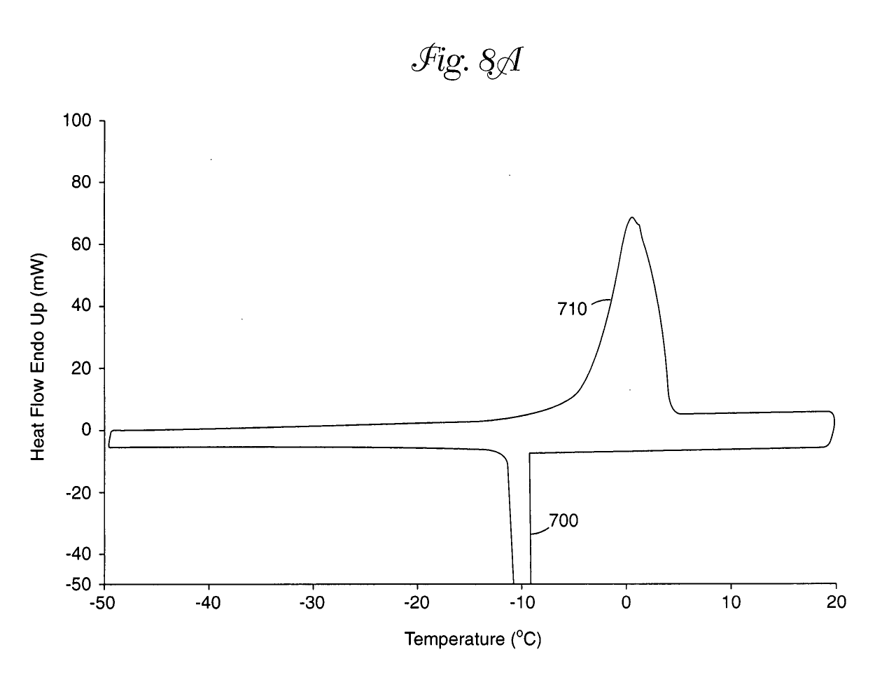

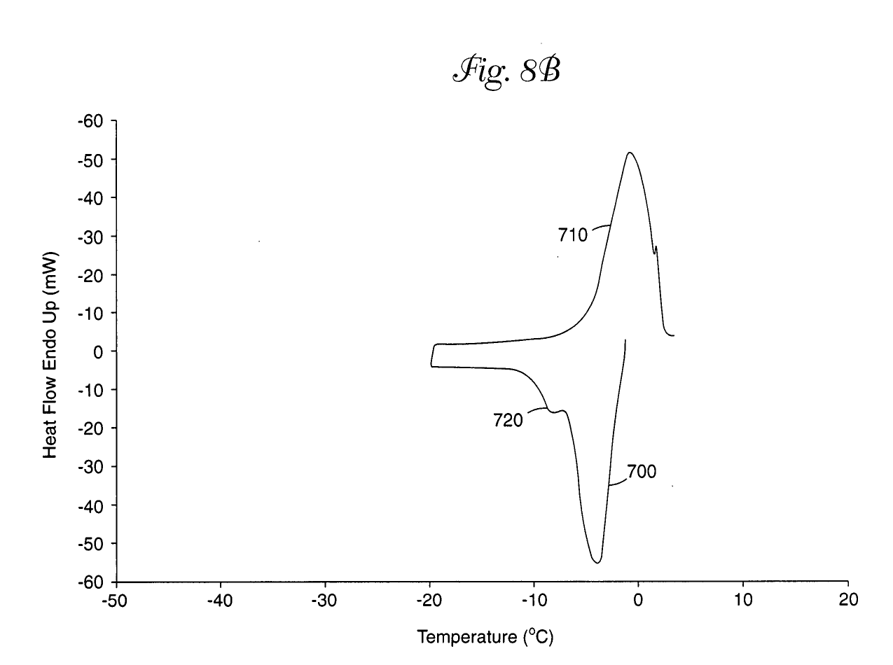

FIGS. 8A and 8B show DSC thermograms of rat liver tissues with/without infusing a half eutectic concentration KNO₃ solution, as described above. FIG. 8A shows the tissue without infusion of the half eutectic concentration of the KNO₃ solution has heat release/absorption peaks, 700 and 710 respectively. These peaks 700 and 710 are associated with water/ice phase change. FIG. 8B, however, shows that when the salt solution is infused (e.g., the half eutectic concentration KNO₃ solution), secondary heat release peak 720 is observed associated with eutectic phase change.

All references identified herein are incorporated in their entirety as if each were incorporated separately. This invention has been described with reference to illustrative embodiments and is not meant to be construed in a limiting sense. Various modifications of the illustrative embodiments, as well as additional embodiments of the invention, will be apparent to persons skilled in the art upon reference to this description.

1-34. (canceled)

35. A method of changing a eutectic freezing point of tissue, comprising:
identifying at least a portion of the tissue to undergo eutectic freezing; and
treating the tissue with a eutectic changing composition for a time, amount and type effective to change the eutectic freezing point of the tissue, wherein the eutectic changing composition does not comprise saline.

36. A method of changing a eutectic freezing point of tissue, comprising:
identifying at least a portion of the tissue to undergo eutectic freezing; and
treating the tissue with a eutectic changing composition for a time, amount and type effective to change the eutectic freezing point of the tissue, wherein the eutectic changing composition comprises hypertonic saline solution.

37. A method of changing a eutectic freezing point of tissue, comprising:
identifying at least a portion of the tissue to undergo eutectic freezing; and
treating the tissue with a eutectic changing composition for a time, amount and type effective to change the eutectic freezing point of the tissue, wherein the eutectic changing composition comprises at least two solutes effective to change a tissue eutectic freezing point at a localized area of the tissue.

38. The method of claim 37, wherein the at least two solutes are selected from the group consisting of KNO₃, KCl, MgSO₄, NaCl, KBr, NH₄Cl, MgCl₂, CaCl₂, glucose, sucrose, and combinations thereof.

39. The method of claim 38 wherein one solute of the at least two solutes is NaCl.

40. A method of cryosurgery, comprising:
identifying tissue to undergo cryosurgery;
treating the tissue with a eutectic changing composition for a time, amount and type effective to change the eutectic freezing point of at least a portion of the tissue, wherein the eutectic changing composition does not comprise saline; and
cooling the tissue at a cooling rate effective to cause a eutectic formation in at least a portion of the tissue.

41. A method of cryosurgery, comprising:
identifying tissue to undergo cryosurgery;
treating the tissue with a eutectic changing composition for a time, amount and type effective to change the eutectic freezing point of at least a portion of the tissue, wherein the eutectic changing composition comprises hypertonic saline; and
cooling the tissue at a cooling rate effective to cause a eutectic formation in at least a portion of the tissue.

42. A method of cryosurgery, comprising:
identifying tissue to undergo cryosurgery;
treating the tissue with a eutectic changing composition for a time, amount and type effective to change the eutectic freezing point of at least a portion of the tissue, wherein the eutectic changing composition comprises at least two solutes effective to change a tissue eutectic freezing point at a localized area of the tissue; and
cooling the tissue at a cooling rate effective to cause a eutectic formation in at least a portion of the tissue.

43. The method of claim 42, wherein the at least two solutes are selected from the group consisting of KNO₃, KCl, MgSO₄, NaCl, KBr, NH₄Cl, MgCl₂, CaCl₂, glucose, sucrose, and combinations thereof.

44. The method of claim 43 wherein one solute of the at least two solutes is NaCl.

45. A method of eutectic formation in tissue, comprising:
treating tissue with a eutectic changing composition for a time, amount and type effective to change the eutectic freezing point of at least a portion of the tissue, wherein the eutectic changing composition does not comprise saline; and
cooling the tissue at a cooling rate effective to cause the eutectic formation in at least a portion of the tissue.

46. A method of eutectic formation in tissue, comprising:
treating tissue with a eutectic changing composition for a time, amount and type effective to change the eutectic freezing point of at least a portion of the tissue, wherein the eutectic changing composition comprises hypertonic saline solution; and
cooling the tissue at a cooling rate effective to cause the eutectic formation in at least a portion of the tissue.

47. A method of eutectic formation in tissue, comprising:
treating tissue with a eutectic changing composition for a time, amount and type effective to change the eutectic freezing point of at least a portion of the tissue, wherein the eutectic changing composition comprises at least two solutes effective to change a tissue eutectic freezing point at a localized area of the tissue; and
cooling the tissue at a cooling rate effective to cause the eutectic formation in at least a portion of the tissue.

48. The method of claim 47, wherein the at least two solutes are selected from the group consisting of KNO₃, KCl, MgSO₄, NaCl, KBr, NH₄Cl, MgCl₂, CaCl₂, glucose, sucrose, and combinations thereof.

49. The method of claim 48 wherein one solute of the at least two solutes is NaCl.

50. A eutectic changing composition, comprising:
a pharmaceutically acceptable solvent; and
at least two solutes in the pharmaceutically acceptable solvent, wherein each solute of the at least two solutes is present at a concentration wherein the combination of the at least two solutes are present in the pharmaceutically acceptable solvent in an amount no greater than a combined eutectic concentration of the at least two solutes.

51. The composition of claim 50 wherein the at least two solutes are selected from the group consisting of KNO₃, KCl, MgSO₄, NaCl, KBr, NH₄Cl, MgCl₂, CaCl₂, glucose, sucrose, and combinations thereof.

52. The composition of claim 51 wherein one solute of the at least two solutes is NaCl.

53. The composition of claim 47 wherein the composition further comprises a contrast agent.

54. The method of claim 35, wherein treating the tissue comprises injecting the eutectic changing composition into the tissue identified to undergo eutectic freezing.

55. The method of claim 36, wherein treating the tissue comprises injecting the eutectic changing composition into the tissue identified to undergo eutectic freezing.

56. The method of claim 37, wherein treating the tissue comprises injecting the eutectic changing composition into the tissue identified to undergo eutectic freezing.

57. The method of claim 40, wherein treating the tissue comprises injecting the eutectic changing composition into the tissue identified to undergo eutectic freezing.

58. The method of claim 41, wherein treating the tissue comprises injecting the eutectic changing composition into the tissue identified to undergo eutectic freezing.

59. The method of claim 42, wherein treating the tissue comprises injecting the eutectic changing composition into the tissue identified to undergo eutectic freezing.

60. The method of claim 45, wherein treating the tissue comprises injecting the eutectic changing composition into the tissue identified to undergo eutectic freezing.

61. The method of claim 46, wherein treating the tissue comprises injecting the eutectic changing composition into the tissue identified to undergo eutectic freezing.

62. The method of claim 47, wherein treating the tissue comprises injecting the eutectic changing composition into the tissue identified to undergo eutectic freezing.
